(19) United States
(12) Patent Application Publication
(10) Pub. No.: US 2020/0163775 A1
(43) Pub. Date: May 28, 2020

(54) FOUR-DIRECTIONAL EXTENDED INTERVERTEBRAL FUSION CAGE DEVICE

(71) Applicant: MANTIZ LOGITECH CO., LTD., Daegu (KR)

(72) Inventors: Eui-Jun KIM, Daegu (KR); Il-Hwan LEE, Busan (KR); Hong-Won YOON, Busan (KR)

(73) Assignee: MANTIZ LOGITECH CO., LTD., Daegu (KR)

(21) Appl. No.: 16/689,661

(22) Filed: Nov. 20, 2019

(30) Foreign Application Priority Data
Nov. 23, 2018 (KR) 10-2018-0146066

(51) Int. Cl. A61F 2/44

(52) U.S. Cl. CPC A61F 2/447; A61F 2002/3051

(57) ABSTRACT

Provided is a four-directional extended intervertebral fusion cage device including a front support, a rear support, and four plates having front ends engaged with the front support and rear ends engaged with the rear support, and configured to slide up in four different directions on inclined surfaces of the front support and the rear support and increase a height and a width of the cage device simultaneously, when the front support and the rear support move toward each other, facing each other.

### CROSS-REFERENCE TO RELATED APPLICATION

This application is based on and claims priority under 35 U.S.C. § 119 to Korean Patent Application No. 10-2018-0146066, filed on Nov. 23, 2018, in the Korean Intellectual Property Office, the disclosure of which is incorporated by reference herein in its entirety.

### BACKGROUND

### 1. Field

The present disclosure relates to a four-directional extended intervertebral fusion cage device, and more particularly, to a four-directional extended intervertebral fusion cage device which facilitates insertion of a cage between adjacent vertebrae and extends the area occupied by the cage, with minimal invasion during a procedure, to stably support the vertebrae.

### 2. Description of Related Art

The traditional open surgery for patient treatment typically has the drawbacks of a large cut area, a large amount of bleeding during the surgery, a patient's slow recovery after the surgery, and a remaining large scar, which may interfere with the patient's future life.

To overcome these drawbacks of the open surgery, a new surgical technique called minimal invasive surgery (MIS) is under development.

MIS is a surgical technique of cutting only a minimal area of a patient's body surface with a long, thin surgical tool specially designed to minimize the required cut area for surgery.

Due to the benefits of a small required cut area, a much smaller amount of bleeding during a procedure than in the open surgery, and hence a patient's fast recovery and a small external scar after the surgery, MIS has been growing in the number of procedures.

The disc between spine bones functions as a joint. As the spine moves, the nucleus pulposus housed inside the disc plays a very important role in minimizing the impact on the spine, while changing its position and shape.

Moisture (water) occupies a major proportion of the nucleus pulposus but decreases in amount with age, resulting in loss of the cushioning function of the disc.

As a consequence, excessive pressure on the fiber causes waist pain. As it progresses further, the fiber stretches or ruptures severely, pressing the back nerve root and thus causing pain in the pelvis and legs.

Since the interval between the spine bones is then gradually narrowed or the vertebral bones settle down, various side effects such as spine deformation occur.

In an approach to treatment of diseases involved in a disc, a damaged intervertebral disc may be removed and the space between two adjacent vertebrae is replaced by a prosthesis such as a cage.

That is, the prosthesis is intended to restore the spinal function by returning the distance between two adjacent vertebral bodies to an original height of the intervertebral disc.

Surgical methods of inserting such an implant in the spine include anterior lumbar interbody fusion (ALIF) in which a prosthesis is inserted from the front of the spine by an open procedure, lateral lumbar interbody fusion (LLIF) in which a prosthesis is inserted through a side, transforaminal lumbar interbody fusion (TLIF) in which a prosthesis is inserted diagonally from a patient's side 30 to 40 mm apart from the center of the back, and posterior lumbar interbody fusion (PLIF) in which a prosthesis is inserted from a patient's back.

Such an example may be Laid-Open Patent No. 10-2015-0048150 entitled “Expandable Intervertebral Cage Assemblies and Methods” (hereinafter referred to as “prior art”).

The prior art discloses a cage body including an upper part with an upper outer surface and a lower part with a lower outer surface, wherein an upper proximal portion is hinged to a lower proximal portion, the upper and lower parts define an internal passage between them, and the upper and lower parts define a distal cavity near a distal end of the respective upper and lower parts, and an expander having a keyed distal portion configured to be received within the distal cavity, wherein the expander is configured to move from a first position, substantially distal from the distal cavity, where the cage body is unexpanded, to a second position, substantially within the cavity, where the cage body is expanded to increase the distance between the upper part outer surface and the lower part outer surface.

In the prior art, as the expander is pulled toward the proximal portion, the expander moves from the first position to the second position, and the hinged upper proximal portion and lower proximal portion increase the distance up and down between the upper distal portion and the lower distal portion. That is, the height of the cage distal portion is increased.

A shortcoming with the prior art lies in that only the height of the cage distal portion, not the height of the proximal portion is controllable, and when the height of the distal portion is controlled, stepwise fine height control is not possible. Moreover, only height control, not width control is allowed.

### SUMMARY

Accordingly, the present disclosure is directed to a four-directional extended intervertebral fusion cage device that substantially obviates one or more problems due to limitations and disadvantages of the related art.

An aspect of the present disclosure devised to overcome the above problem is to provide a four-directional extended intervertebral fusion cage device which enables four plates to slide up in four different directions on inclined surfaces and rails formed on a front support and a rear support by narrowing the distance between the front support and the rear support in an intervertebral fusion cage for a lumbar vertebra, a cervical spine, a vertebral body, or the like, to simultaneously increase the height and width of the cage device, and which enables the four plates to slide down on the inclined surfaces and the rails formed on the front support and the rear support by broadening the distance between the front support and the rear support, to simultaneously decrease the height and width of the cage device.

Additional advantages, objects, and features of the disclosure will be set forth in part in the description which follows and in part will become apparent to those having ordinary skill in the art upon examination of the following or may be learned from practice of the disclosure. The objectives and other advantages of the disclosure may be realized and attained by the structure particularly pointed out in the written description and claims hereof as well as the appended drawings.

In an aspect of the present disclosure, a four-directional extended intervertebral fusion cage device includes a front support, a rear support, and four plates having front ends engaged with the front support and rear ends engaged with the rear support, and configured to slide up in four different directions on inclined surfaces of the front support and the rear support and increase a height and a width of the cage device simultaneously, when the front support and the rear support move toward each other, facing each other.

The front support may include a body on which four inclined surfaces are formed, and four rails protruding from the four inclined surfaces, respectively, and the rear support may include a body on which four inclined surfaces are formed, and four rails protruding from the four inclined surfaces, respectively. The inclined surfaces formed on the front support and the rear support may be inclined toward the four plates located at a center.

The four plates may be disposed at upper left, upper right, lower left, and lower right positions, respectively, and each of the four plates may include a front engagement portion including an inclined groove recessed inward, to enable the plate to slide on the front support in engagement with an inclined surface and a rail of the front support, and a rear engagement portion including an inclined groove recessed inward, to enable the plate to slide on the rear support in engagement with an inclined surface and a rail of the rear support.

Further, to prevent slip-off of the four plates from the rails during sliding movement on the front support and the rear support, each of the rails of the front support and the rear support may include an engagement groove recessed along a length direction of the rail, and a stepped protrusion protruding inside an inclined groove recessed inward on a plate.

Further, the four-directional extended intervertebral fusion cage device may further include a cylindrical bolt coupler disposed at a rear end of the front support and having threads formed on an inner circumferential surface thereof, and a bolt passing through a through hole formed at a center of the rear support, coupled with the bolt coupler, and configured to enable the four plates to slide up on the inclined surfaces and move away from each other by moving forward according to a rotation direction and narrowing a distance between the front support and the rear support, and enable the four plates to slide down on the inclined surfaces and move toward each other by moving backward and broadening the distance between the front support and the rear support.

The four plates may be disposed at upper left, upper right, lower left, and lower right positions, respectively, and include a first vertical pin coupler having a top engaged with the upper left plate and a bottom engaged with the lower left plate, and configured to guide the upper left plate and the lower left plate to move away from each other, when the height of the cage device is increased, a second vertical pin coupler having a top engaged with the upper right plate and a bottom engaged with the lower right plate, and configured to guide the upper right plate and the lower right plate to move away from each other, when the height of the cage device is increased, a first horizontal pin coupler having one portion engaged with the upper left plate and the other portion engaged with the upper right plate, and configured to guide the upper left plate and the upper right plate to move away from each other, when the width of the cage device is increased, and a second horizontal pin coupler having one portion engaged with the lower left plate and the other portion engaged with the lower right plate, and configured to guide the lower left plate and the lower right plate to move away from each other, when the width of the cage device is increased. When the front support and the rear support move toward each other, facing each other, the four plates may be extended in height by the first vertical pin coupler and the second vertical pin coupler, and in width by the first horizontal pin coupler and the second horizontal pin coupler.

The four-directional extended intervertebral fusion cage device may further include a tractor extended forward from left and right sides of the rear support, a mover connecting between left and right sides of the tractor and including a through hole having threads formed on an inner circumferential surface thereof, and a ball screw passing through the through hole of the mover, engaged with a rear end of the front support, and configured to enable the four plates to slide up on the inclined surfaces and move away from each other by moving the mover forward according to a rotation direction and narrowing a distance between the front support and the rear support, and enable the four plates to slide down on the inclined surfaces and move toward each other by moving the mover backward and broadening the distance between the front support and the rear support.

The four plates may be disposed at upper left, upper right, lower left, and lower right positions, respectively, and include a first vertical plate coupler shaped into a plate, extended from the lower or upper left plate, and configured to guide the upper left plate and the lower left plate to move away from each other, in engagement with the groove formed on the lower or upper left plate, when the height of the cage device is increased, a second vertical plate coupler shaped into a plate, extended from the upper or lower right plate, and configured to guide the upper right plate and the lower right plate to move away from each other, in engagement with the groove formed on the lower or upper right plate, when the height of the cage device is increased, a first horizontal plate coupler shaped into a plate, extended from the upper left or right plate, and configured to guide the upper left plate and the upper right plate to move away from each other, in engagement with the groove formed on the upper right or left plate, when the width of the cage device is increased, and a second horizontal plate coupler shaped into a plate, extended from the lower left or right plate, and configured to guide the lower left plate and the lower right plate to move away from each other, in engagement with the groove formed on the lower right or left plate, when the width of the cage device is increased.

It is to be understood that both the foregoing general description and the following detailed description of the present disclosure are exemplary and explanatory and are intended to provide further explanation of the disclosure as claimed.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIGS. 1A and 1B are diagrams illustrating a four-directional extended intervertebral fusion cage device viewed from various angles before its height and width are increased according to a preferred embodiment of the present disclosure, and the four-directional extended intervertebral fusion cage device viewed from various angles after its height and width are increased according to the preferred embodiment of the present disclosure;

FIG. 2 is an exploded perspective view illustrating the four-directional extended intervertebral fusion cage device according to the preferred embodiment of the present disclosure;

FIG. 3 is a diagram illustrating a front support viewed from various angles in the four-directional extended intervertebral fusion cage device according to the preferred embodiment of the present disclosure;

FIG. 4 is a diagram illustrating a rear support viewed from various angles in the four-directional extended intervertebral fusion cage device according to the preferred embodiment of the present disclosure;

FIG. 5 is a diagram illustrating a plate viewed from various angles in the four-directional extended intervertebral fusion cage device according to the preferred embodiment of the present disclosure;

FIG. 6 is a diagram illustrating the four-directional extended intervertebral fusion cage device having an increased height and width, with an upper right plate removed to show an internal structure according to the preferred embodiment of the present disclosure;

FIGS. 7A and 7B are diagrams illustrating the four-directional extended intervertebral fusion cage device with two upper plates removed before its height and width are increased, viewed from various angles according to the preferred embodiment of the present disclosure, and illustrating the four-directional extended intervertebral fusion cage device with the two upper plates removed after its height and width are increased, viewed from various angles according to the preferred embodiment of the present disclosure;

FIG. 8 is a diagram illustrating plates which slide up on inclined surfaces and rails of the front support and the rear support and thus are extended by rotating a bolt to move forward and thus narrowing the distance between the front support and the rear support in the four-directional extended intervertebral fusion cage device according to the preferred embodiment of the present disclosure;

FIG. 9 is a diagram illustrating engagement between four plates and pins in the four-directional extended intervertebral fusion cage device according to the preferred embodiment of the present disclosure;

FIGS. 10A and 10B are diagrams illustrating a four-directional extended intervertebral fusion cage device viewed from various angles before its height and width are increased according to another embodiment of the present disclosure, and illustrating the four-directional extended intervertebral fusion cage device viewed from various angles after its height and width are increased according to another embodiment of the present disclosure;

FIG. 11 is an exploded perspective view illustrating the four-directional extended intervertebral fusion cage device according to another embodiment of the present disclosure;

FIG. 12 is a diagram illustrating a plate viewed from various angles in the four-directional extended intervertebral fusion cage device according to another embodiment of the present disclosure;

FIG. 13 is a diagram illustrating plates which slide up on inclined surfaces and rails of the front support and the rear support and thus are extended by rotating a bolt to move forward and thus narrowing the distance between the front support and the rear support in the four-directional extended intervertebral fusion cage device according to another embodiment of the present disclosure; and

FIG. 14 is a diagram illustrating engagement between four plates and pins in the four-directional extended intervertebral fusion cage device according to another embodiment of the present disclosure.

### DETAILED DESCRIPTION

Reference will now be made in detail to the preferred embodiments of the present disclosure, examples of which are illustrated in the accompanying drawings. Wherever possible, the same reference numbers will be used throughout the drawings to refer to the same or like parts.

The advantages and features of the present disclosure and methods of achieving them will become apparent from embodiments described below in conjunction with the attached drawings.

However, the present disclosure may be implemented in various ways, not limited to the disclosed embodiments.

In the disclosure, the present embodiment makes the disclosure complete, and is provided to indicate the comprehensive scope of the present disclosure to those skilled in the art.

The present disclosure is defined by the scope of the appended claims.

Accordingly, known components, operations, and techniques will not be described in detail in some embodiments to avoid ambiguous interpretation of the present disclosure.

Throughout the specification, like reference numerals denote the same components, and terms used (mentioned) herein are intended to describe the embodiments, not limiting them.

Unless otherwise specified in the context, a singular form includes plural referents, and the term “include (or comprised of)” signifies the presence of a component and an operation, not excluding the presence or addition of one or more other components and operations.

Unless otherwise defined, all terms (including technical or scientific terms) used in the disclosure may have the same meanings as generally understood by those skilled in the art.

Unless otherwise defined, the terms as generally defined in dictionaries should not be interpreted as ideally or excessively formal meanings.

Now, a description will be given of a preferred embodiment of the present disclosure with reference to the attached drawings.

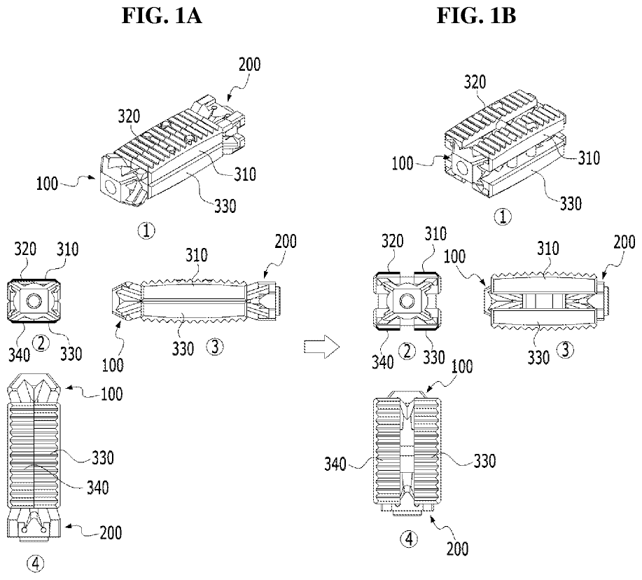

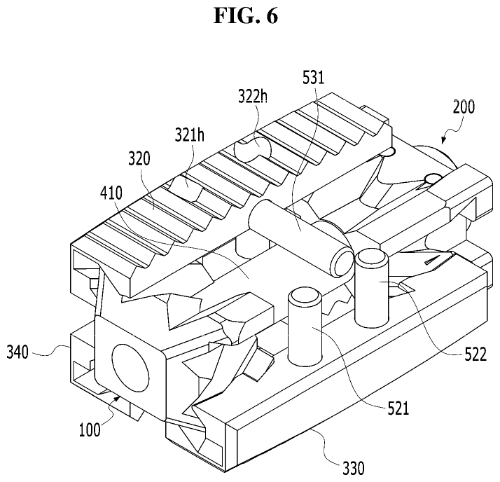

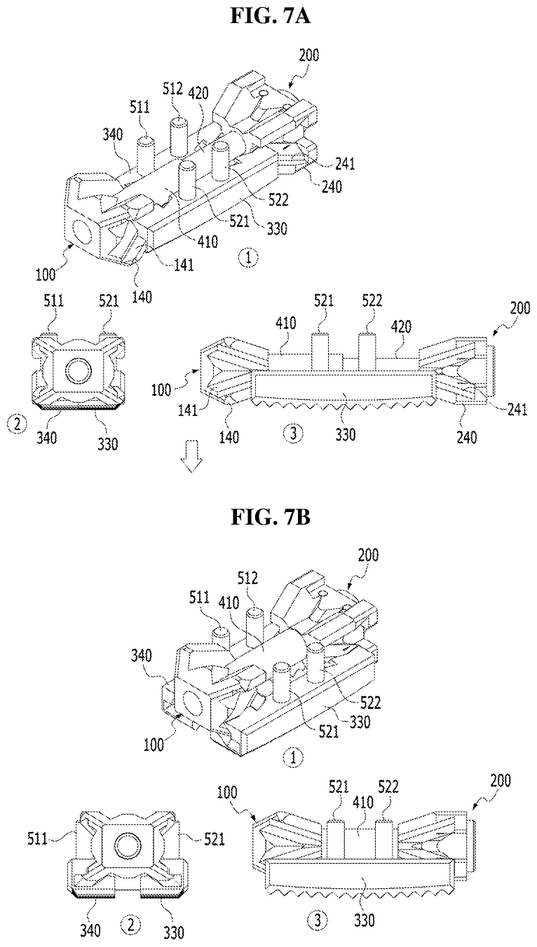

The present disclosure will be described with reference to FIGS. 1A to 9.

A four-directional extended intervertebral fusion cage device according to the present disclosure includes a front support 100, a rear support 200, and four plates 310, 320, 330, and 340.

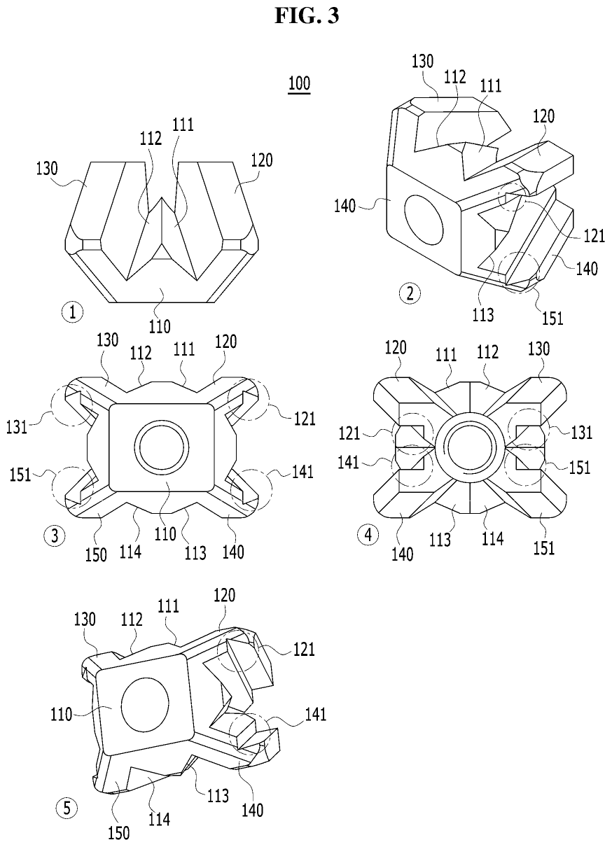

Referring to FIG. 3, the front support 100 is a part which is inserted first between two spines of a patient during a procedure. The front support 100 is formed in such a structure that the cross-section of a body with four surfaces becomes smaller from front to back, that is, in an inclined structure. However, in order to facilitate smooth insertion of a cage into a human body, a forward inclined surface is formed on a predetermined front part of the front support 100.

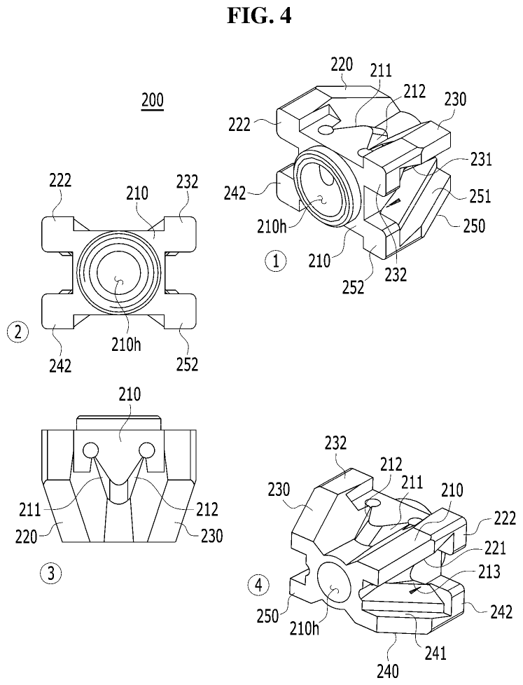

Referring to FIG. 4, the rear support 200 is the last part which is inserted into the human body during the procedure, and with which an insertion instrument is engaged. The rear support 200 is formed by cutting its body to be inclined, like the front support 100, and has a cross-section which becomes smaller from back to front, that is, an inclined structure, unlike the front support 100.

The four plates 310, 320, 330, and 340 have front ends engaged with the front support 100, and rear ends engaged with the rear support 200. When the front support 100 and the rear support 200 move toward each other, facing each other, the four plates 310, 320, 330, and 340 slide up in four different directions on inclined surfaces and rails of the front support 100 and the rear support 200, thereby increasing the height and width of the cage device at the same time.

On the contrary, when the front support 100 and the rear support 200 move away from each other, the four plates 310, 320, 330, and 340 slide down on the inclined surfaces and rails of the front support 100 and the rear support 200, thereby decreasing the height and width of the cage device at the same time.

FIG. 1A illustrates the cage device before the front support 100 and the rear support 200 move toward each other, facing each other, that is, before the height and width of the cage device are increased.

In FIG. 1B, as the front support 100 and the rear support 200 move toward each other, facing each other, the front ends of the plates 310, 320, 330, and 340 slid up on the inclined surfaces and rails of the front support 100, and the rear ends of the plates 310, 320, 330, and 340 slid up on the inclined surfaces and the rails of the rear support 200, thereby increasing the height and width of the cage device at the same time.

Referring to FIG. 3, the front support 100 includes a body 110 on which four inclined surfaces 111 to 114 are formed, and four rails 120, 130, 140, and 150 protruding respectively from the four inclined surfaces 111 to 114.

Referring to FIG. 4, the rear support 200 includes a body 210 on which four inclined surfaces 211 to 214 are formed, and four rails 220, 230, 240, and 250 protruding respectively from the four inclined surfaces 211 to 214.

The inclined surfaces 111 to 114 and 211 to 214 formed on the front support 100 and the rear support 200 may be formed to be inclined toward the four plates 310, 320, 330, and 340 located at the center. That is, the inclined surfaces 111 to 114 and 211 to 214 formed on the front support 100 and the rear support 200 are inclined in a facing direction.

The four plates 310, 320, 330, and 340 are disposed respectively at upper left, upper right, lower left, and lower right positions. The four plates 310, 320, 330, and 340 are provided with front engagement portions having inclined grooves recessed inward to move along the front support 100 in engagement with the inclined surfaces 111 to 114 and the rails 120, 130, 140, and 150 of the front support 100 and rear engagement portions having inclined grooves recessed inward to move along the rear support 200 in engagement with the inclined surfaces 211 to 214 and the rails 220, 230, 240, and 250 of the rear support 200.

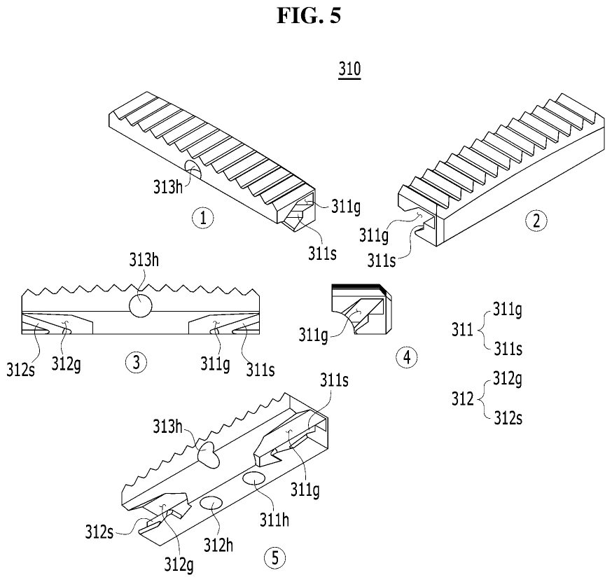

Reference is made to FIG. 5 which illustrates the upper right plate 310 viewed from various angles. A front engagement portion 311 includes an inclined groove 311g recessed inward, which is engaged with the rail 120 of the front support 100. A rear engagement portion 312 includes an inclined groove 312g recessed inward, which is engaged with the rail 220 of the rear support 200.

To prevent slip-off of the four plates 310, 320, 330, and 340 from the rails during sliding movement on the front support 100 and the rear support 200, each of the rails of the front support 100 and the rear support 200 includes an engagement groove recessed along a length direction of the rail, and a stepped protrusion protruding inside the inclined groove recessed inward on a plate 310, 320, 330, or 340.

Referring to FIG. 3, engagement grooves 121, 131, 141, and 151 are formed in the shape of ‘ [symbol] ’ or ‘ [symbol] ’ in a length direction in the four rails 120, 130, 140, and 150 of the front support 100.

Referring to FIG. 4, engagement grooves 221, 231, 241, and 251 are formed in the shape of ‘ [symbol] ’ or ‘ [symbol] ’ in a length direction in the four rails 220, 230, 240, and 250 of the second support 200.

Referring to FIG. 5, the front engagement portion 311 of the plate 310 includes a stepped protrusion 311s protruding inside the inclined groove 311g recessed inward. The stepped protrusion 311s is engaged with the engagement groove 121 recessed along the length direction in the rail 120 of the front support 100.

The rear engagement portion 312 of the plate 310 includes a stepped protrusion 312s protruding inside the inclined groove 312g recessed inward. The stepped protrusion 312s is engaged with the engagement groove 221 recessed along the length direction in the rail 220 of the rear support 200.

This engagement structure may prevent the plates 310, 320, 330, and 340 from slipping off from the rails 120, 130, 140, and 150 of the front support 100 and the rails 220, 230, 240, and 250 of the rear support 200 during sliding up or down on the rails.

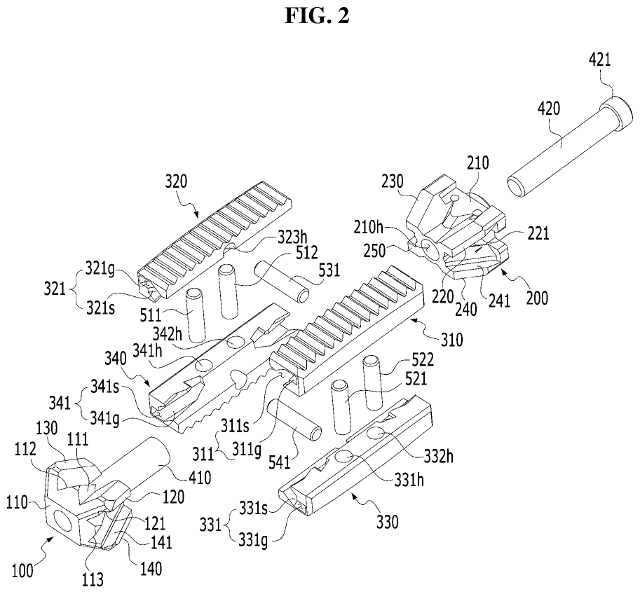

Referring to FIGS. 2, 7A-7B and 8, a cylindrical bolt coupler 410 is disposed at a rear end of the front support 100, and has threads formed on its inner circumferential surface.

A bolt 420 passes through a through hole 210h formed at the center of the rear support 200 and is engaged inside the bolt coupler 410. As the bolt 420 moves forward according to a rotation direction, the bolt 420 narrows the distance between the front support 100 and the rear support 200, so that the four plates 310, 320, 330, and 340 slide up on the inclined surfaces, farther from each other.

Alternatively, as the bolt 420 moves backward, the bolt 420 broadens the distance between the front support 100 and the rear support 200, so that the four plates 310, 320, 330, and 340 slide down on the inclined surfaces, nearer to each other.

The bolt 420 has threads formed thereon, which are engaged with the threads on the inner circumferential surface of the bolt coupler 410, and moves forward or backward according to a rotation direction of the bolt 420. A head 421 formed at a rear end of the bolt 420 is engaged with the through hole 210h of the rear support 200 from behind the through hole 210h. The head 421 moves the rear support 200 forward along with forward movement of the bolt 420 and moves the rear support 200 backward along with backward movement of the bolt 420.

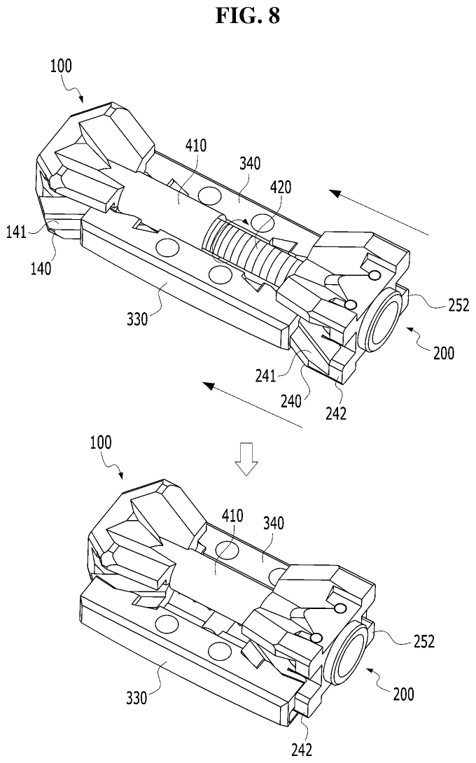

Referring to FIGS. 4 and 8, the four rails 220, 230, 240, and 250 of the rear support 200 are provided, at the rear ends thereof, with stoppers 222, 232, 242, and 252, respectively, which are formed perpendicularly to the length direction of the rails 220, 230, 240, and 250.

When the rear support 200 moves forward, the four plates 310, 320, 330, and 340 slide up on the rails 120, 130, 140, and 150 of the front support 100 and the rails 220, 230, 240, and 250 of the rear support 200, with a gradually increased height and width, and the rear ends of the four plates 310, 320, 330, and 340 are stopped by the stoppers 222, 232, 242, and 252, thereby prohibiting further forward movement of the rear support 200 and a further increase of the height and width of the four plates 310, 320, 330, and 340.

Referring to FIGS. 2, 7A-7B and 9, the cage device includes first vertical pin couplers 511 and 512, second vertical pin couplers 521 and 522, a first horizontal pin coupler 531, and a second horizontal pin coupler 541.

In the drawings, only two first vertical pin couplers 511 and 512 and only two second vertical pin couplers 521 and 522 are shown, which should not be construed as limiting the present disclosure.

The tops of the first vertical pin couplers 511 and 512 are coupled with pin coupling holes 321h and 322h formed on the upper left plate 320, and the bottoms of the first vertical pin couplers 511 and 512 are coupled with pin coupling holes 341h and 342h formed on the lower left plate 340, so as to guide the upper left plate 320 and the lower left plate 340 to vertically move away from each other, as the height of the cage device increases.

The tops of the second vertical pin couplers 521 and 522 are coupled with pin coupling holes 311h and 312h formed on the upper right plate 310, and the bottoms of the second vertical pin couplers 521 and 522 are coupled with pin coupling holes 331h and 332h formed on the lower right plate 330, so as to guide the upper right plate 310 and the lower right plate 330 to vertically move away from each other, as the height of the cage device increases.

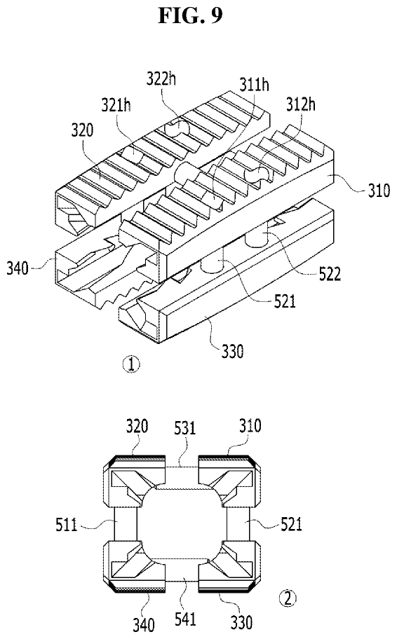

Referring to FIG. 9, the upper left plate 320 moves up and down along the first vertical pin couplers 511 and 512, and the upper right plate 310 moves up and down along the second vertical pin couplers 521 and 522, with the lower left plate 340 and the lower right plate 330 stationary. That is, although the lower left plate 340 and the lower right plate 330 do not move, the upper left plate 320 and the upper right plate 310 move up and down, thereby increasing and decreasing the height of the cage device.

The first horizontal pin coupler 531 has one end engaged with a pin coupling hole 323h formed on a side surface of the upper left plate 320 and the other end engaged with a pin coupling hole formed on a side surface of the upper right plate 310, so as to guide the upper left plate 320 and the upper right plate 310 to horizontally move away from each other, as the width of the cage device increases.

The second horizontal pin coupler 541 has one end engaged with a pin coupling hole formed on a side surface of the lower left plate 340 and the other end engaged with a pin coupling hole formed on a side surface of the lower right plate 330, so as to guide the lower left plate 340 and the lower right plate 330 to horizontally move away from each other, as the width of the cage device increases.

As noted from the above description, when the front support 100 and the rear support 200 move toward each other, facing each other, the four plates 310, 320, 330, and 340 are extended in height by the first vertical pin couplers 511 and 512 and the second vertical pin couplers 521 and 522, and in width by the first horizontal pin coupler 531 and the second horizontal pin coupler 541.

With reference to the attached drawings, another embodiment of the present disclosure will be described.

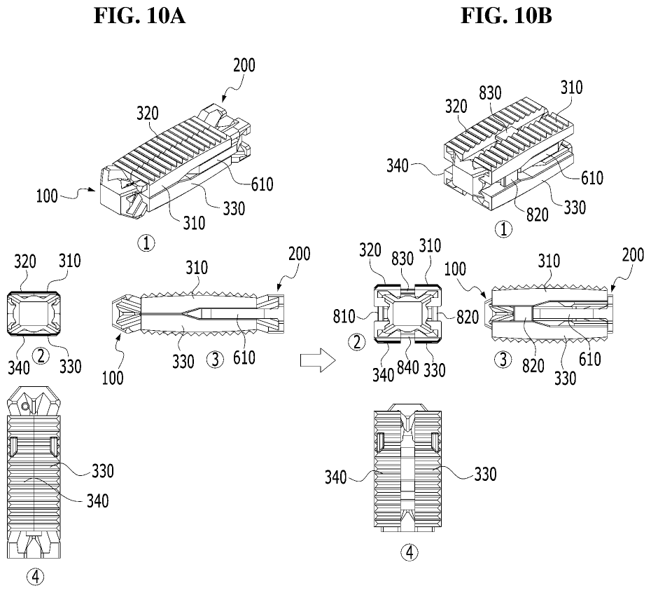

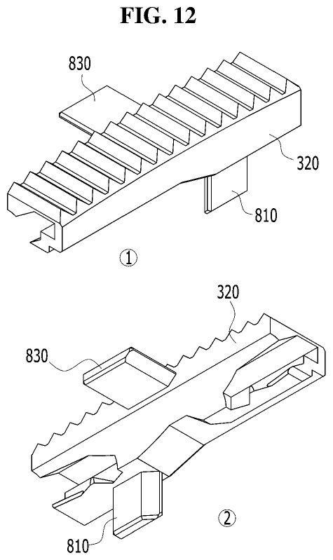

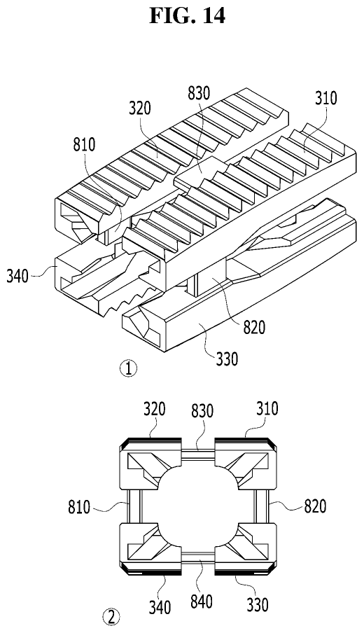

A four-directional extended fusion cage device according to another embodiment of the present disclosure will be described with reference to FIGS. 10A to 14. The same part as in the preferred embodiment of the present disclosure described before with reference to FIGS. 1A to 9 will not be described herein to avoid redundancy.

The four-directional extended fusion cage device according to another embodiment of the present disclosure includes the front support 100, the rear support 200, and the four plates 310 to 340, and further includes a tractor 600, a mover 630, a ball screw 700, a first vertical plate coupler 810, a second vertical plate coupler 820, a first horizontal plate coupler 830, and a second horizontal plate coupler 840.

The tractor 600 includes a left traction portion 620 and a right traction portion 610 which are extended forward from the left and right sides of the rear support 200.

The mover 630 connects the front ends of the left traction portion 620 and the right traction portion 610 of the tractor 600, and includes a through hole 630h having threads formed on the outer circumferential surface thereof.

The ball screw 700 passes through the through hole 630h of the mover 630 and is coupled with the rear end of the front support 100. The ball screw 700 moves the mover 630 forward according to a rotation direction, narrowing the distance between the front support 100 and the rear support 200, so that the four plates 310, 320, 330, and 340 slide up on the inclined surfaces, farther from each other. Alternatively, the ball screw 700 moves the mover 630 backward, broadening the distance between the front support 100 and the rear support 200, so that the four plates 310, 320, 330, and 340 slide down on the inclined surfaces, nearer to each other.

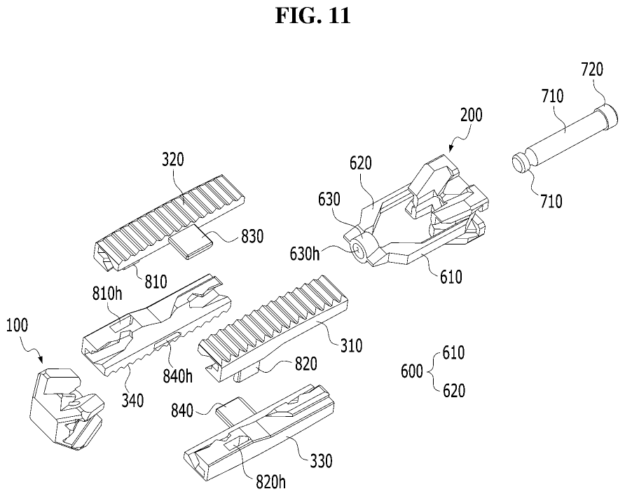

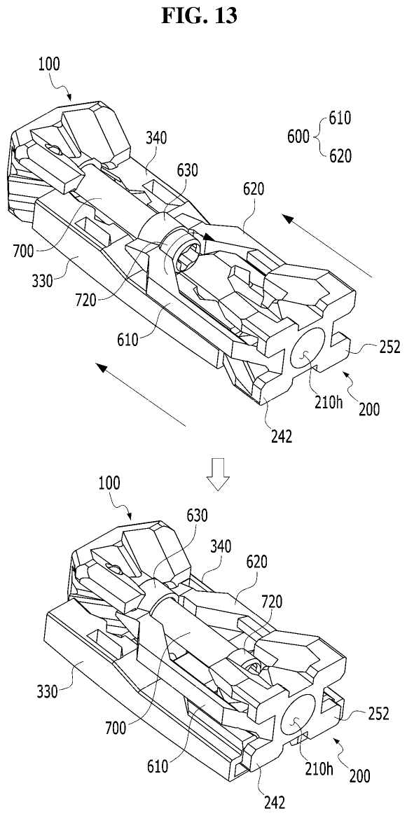

Referring to FIGS. 11 and 13, a front end 710 of the ball screw 700 is coupled with the rear end of the front support 100, and a head 720 formed at the rear end of the ball screw 700 is located behind the mover 630 to prevent slip-off of the mover 630 from the ball screw 700.

When the ball screw 700 is rotated by means of a tool, the mover 630 moves forward on the ball screw 700, thus moving the rear support 200 forward, or moves backward on the ball screw 700, thus moving the rear support 200 backward.

The first vertical plate coupler 810, which is shaped into a plate, is extended from the upper or lower left plate 320 and coupled with a groove 810h formed on the lower or upper left plate 340. Therefore, as the height of the cage device increases, the first vertical plate coupler 810 guides the upper left plate 320 and the lower left plate 340 to vertically move away from each other.

The second vertical plate coupler 820, which is shaped into a plate, is extended from the upper or lower right plate 310 and coupled with a groove 820h formed on the lower or upper right plate. Therefore, as the height of the cage device increases, the second vertical plate coupler 820 guides the upper right plate 310 and the lower right plate 330 to vertically move away from each other.

The first horizontal plate coupler 830, which is shaped into a plate, is extended from the upper left or right plate 320 and coupled with a groove formed on the upper right or left plate 310. Therefore, as the width of the cage device increases, the first horizontal plate coupler 830 guides the upper left plate 320 and the upper right plate 310 to horizontally move away from each other.

The second horizontal plate coupler 840, which is shaped into a plate, is extended from the lower left or right plate 330 and coupled with a groove 840h formed on the lower right or left plate 340. Therefore, as the width of the cage device increases, the second horizontal plate coupler 840 guides the lower left plate 340 and the lower right plate 330 to horizontally move away from each other.

As such, the use of plates leads to the same functions and effects as the use of pins as described above.

As described above, the basic technical idea of the present disclosure is to provide a four-directional extended intervertebral fusion cage in which four plates slide up in four different directions on inclined surfaces and rails formed on a front support and a rear support by narrowing the distance between the front support and the rear support in the intervertebral fusion cage for a lumbar vertebra, a cervical spine, a vertebral body, or the like, to simultaneously increase the height and width of the cage device, and the four plates slide down on the inclined surfaces and the rails formed on the front support and the rear support by broadening the distance between the front support and the rear support, to simultaneously decrease the height and width of the cage device.

As is apparent from the foregoing description, compared to an existing intervertebral fusion cage for which only one of a height and a width is controllable, the four-directional extended intervertebral fusion cage device of the present disclosure may increase and decrease a height and a width simultaneously.

Because the distance between a front support and a rear support is narrowed or broadened according to a rotation direction of the device by enabling four plates located between the front support and the rear support to naturally move along inclined surfaces and rails formed on the front support and the rear support without using an additional complex instrument for increasing or decreasing a height and a width simultaneously, fine height and width control is possible and the durability of the cage device may be increased.

Further, a complex procedure instrument is not used, the cage itself is simple in structure, and a time taken for a procedure may be significantly shortened.

It will be apparent to those skilled in the art that various modifications and variations can be made in the present disclosure without departing from the spirit or scope of the disclosures. Thus, it is intended that the present disclosure covers the modifications and variations of this disclosure provided they come within the scope of the appended claims and their equivalents.

### DESCRIPTION OF REFERENCE NUMERALS

100 . . . front support
110: body
120, 130, 140, 150, 220, 230, 240, 250 . . . rail
111 to 114 . . . inclined surface
200 . . . rear support
211 to 214 . . . inclined surface
310, 320, 330, 340 . . . plate
410 . . . bolt coupler
420 . . . bolt
511, 512 . . . first vertical pin coupler
521, 522 . . . second vertical pin coupler
531 . . . first horizontal pin coupler
541 . . . second horizontal pin coupler
600 . . . tractor
630 . . . mover
700 . . . ball screw
810 . . . first vertical plate coupler
820 . . . second vertical plate coupler
830 . . . first horizontal plate coupler
840 . . . second horizontal plate coupler

What is claimed is:

1. A four-directional extended intervertebral fusion cage device comprising:
a front support;
a rear support; and
four plates having front ends engaged with the front support and rear ends engaged with the rear support, and configured to slide up in four different directions on inclined surfaces of the front support and the rear support and increase a height and a width of the cage device simultaneously, when the front support and the rear support move toward each other, facing each other.

2. The four-directional extended intervertebral fusion cage device according to claim 1, wherein the front support comprises:
a body on which four inclined surfaces are formed; and
four rails protruding from the four inclined surfaces, respectively,
wherein the rear support comprises:
a body on which four inclined surfaces are formed; and
four rails protruding from the four inclined surfaces, respectively, and
wherein the inclined surfaces formed on the front support and the rear support are inclined toward the four plates located at a center.

3. The four-directional extended intervertebral fusion cage device according to claim 2, wherein the four plates are disposed at upper left, upper right, lower left, and lower right positions, respectively, and
wherein each of the four plates comprises:
a front engagement portion including an inclined groove recessed inward, to enable the plate to slide on the front support in engagement with an inclined surface and a rail of the front support; and
a rear engagement portion including an inclined groove recessed inward, to enable the plate to slide on the rear support in engagement with an inclined surface and a rail of the rear support.

4. The four-directional extended intervertebral fusion cage device according to claim 3, wherein each of the rails of the front support and the rear support comprises:
an engagement groove recessed along a length direction of the rail; and
a stepped protrusion protruding inside an inclined groove recessed inward on a plate, to prevent slip-off of the four plates from the rails during sliding movement on the front support and the rear support.

5. The four-directional extended intervertebral fusion cage device according to claim 1, further comprising:
a cylindrical bolt coupler disposed at a rear end of the front support and having threads formed on an inner circumferential surface thereof; and
a bolt passing through a through hole formed at a center of the rear support, coupled with the bolt coupler, and configured to enable the four plates to slide up on the inclined surfaces and move away from each other by moving forward according to a rotation direction and narrowing a distance between the front support and the rear support, and enable the four plates to slide down on the inclined surfaces and move toward each other by moving backward and broadening the distance between the front support and the rear support.

6. The four-directional extended intervertebral fusion cage device according to claim 1, wherein the four plates are disposed at upper left, upper right, lower left, and lower right positions, respectively, and comprise:
a first vertical pin coupler having a top engaged with the upper left plate and a bottom engaged with the lower left plate, and configured to guide the upper left plate and the lower left plate to move away from each other, when the height of the cage device is increased;
a second vertical pin coupler having a top engaged with the upper right plate and a bottom engaged with the lower right plate, and configured to guide the upper right plate and the lower right plate to move away from each other, when the height of the cage device is increased;
a first horizontal pin coupler having one portion engaged with the upper left plate and the other portion engaged with the upper right plate, and configured to guide the upper left plate and the upper right plate to move away from each other, when the width of the cage device is increased; and
a second horizontal pin coupler having one portion engaged with the lower left plate and the other portion engaged with the lower right plate, and configured to guide the lower left plate and the lower right plate to move away from each other, when the width of the cage device is increased,
wherein when the front support and the rear support move toward each other, facing each other, the four plates are extended in height by the first vertical pin coupler and the second vertical pin coupler, and in width by the first horizontal pin coupler and the second horizontal pin coupler.

7. The four-directional extended intervertebral fusion cage device according to claim 1, further comprising:
a tractor extended forward from left and right sides of the rear support;
a mover connecting between left and right sides of the tractor and including a through hole having threads formed on an inner circumferential surface thereof; and
a ball screw passing through the through hole of the mover, engaged with a rear end of the front support, and configured to enable the four plates to slide up on the inclined surfaces and move away from each other by moving the mover forward according to a rotation direction and narrowing a distance between the front support and the rear support, and enable the four plates to slide down on the inclined surfaces and move toward each other by moving the mover backward and broadening the distance between the front support and the rear support.

8. The four-directional extended intervertebral fusion cage device according to claim 1, wherein the four plates are disposed at upper left, upper right, lower left, and lower right positions, respectively, and comprise:
a first vertical plate coupler shaped into a plate, extended from the lower or upper left plate, and configured to guide the upper left plate and the lower left plate to move away from each other, in engagement with the groove formed on the lower or upper left plate, when the height of the cage device is increased;
a second vertical plate coupler shaped into a plate, extended from the upper or lower right plate, and configured to guide the upper right plate and the lower right plate to move away from each other, in engagement with the groove formed on the lower or upper right plate, when the height of the cage device is increased;
a first horizontal plate coupler shaped into a plate, extended from the upper left or right plate, and configured to guide the upper left plate and the upper right plate to move away from each other, in engagement with the groove formed on the upper right or left plate, when the width of the cage device is increased; and
a second horizontal plate coupler shaped into a plate, extended from the lower left or right plate, and configured to guide the lower left plate and the lower right plate to move away from each other, in engagement with the groove formed on the lower right or left plate, when the width of the cage device is increased.
